(19) United States
(12) Patent Application Publication
(10) Pub. No.: US 2025/0189522 A1
(43) Pub. Date: Jun. 12, 2025

(54) ENHANCEMENT COMPOSITIONS AND USES THEREOF

(71) Applicant: Yuriy Markushin, Dover, DE (US)

(72) Inventor: Yuriy Markushin, Dover, DE (US)

(73) Assignee: DELAWARE STATE UNIVERSITY, DOVER, DE (US)

(21) Appl. No.: 18/844,487

(22) PCT Filed: Mar. 17, 2023
(86) PCT No.: PCT/US2023/015474

§ 371 Date: Sep. 6, 2024

Related U.S. Application Data
(60) Provisional application No. 63/320,758, filed on Mar. 17, 2022.

(51) Int. Cl. G01N 33/543; B01L 3/02

(52) U.S. Cl. CPC G01N 33/54393; B01L 3/0217; G01N 2333/765

(57) ABSTRACT

The present invention provides a method for detecting an analyte in a liquid test sample. The detection method comprises mixing the test sample with a liquid enhancement composition to make a mixture, and contacting the mixture with a binding agent capable of forming a complex with the analyte. The enhancement composition may comprise one or more non-ionic amphiphilic polymeric compounds. Also provided is a mixing device comprising a first compartment comprising a liquid test sample; a second compartment comprising a liquid enhancement composition; and a third compartment in which the test sample from the first compartment and the enhancement composition from the second compartment are mixed to make a mixture.

### CROSS-REFERENCE TO RELATED APPLICATION

This application claims priority to U.S. Provisional Application No. 63/320,758 filed Mar. 17, 2022, the contents of which are incorporated herein by reference in their entireties for all purposes.

### FIELD OF THE INVENTION

The invention relates generally to an enhancement composition and use thereof for detecting analytes in a liquid sample, for example, a biological sample.

### BACKGROUND OF THE INVENTION

Lateral flow assays (LFA) or strip tests are known in art as rapid and portable point-of-care tests due to its simplicity and low cost. LFA-based tests are widely used in medical field for qualitative, semiquantitative and to some extent quantitative detection of specific biomarkers of interest, such as antibodies, antigens and other molecules-biomarkers related to health condition or decrease in varieties of biological samples including blood, saliva, sweat, urine, human feces, and others. Besides from the medical field, LFA-based tests are also widely employed in veterinary, food production, water safety among other areas of application. At the same time, LFA are also commonly known in art as tests having typically poor limits of detection of molecules of interest in comparison with more complicated laboratory-based methods.

The working principle of the LFA is known in art. According to that principle, a sample in liquid form is delivered to the portion of LFA, called the adsorbent sample pad that might contain some amount of salts and surfactants, and then allowed to move forward through the following conjugate release pad, that contains the antibodies specific to molecules of interest to be detected in a sample; and those antibodies are conjugated to colloidal gold, carbon or latex colored nanoparticles particles. After that, the sample in a mixture with conjugated to nanoparticles antibodies are moving along the strip test porous membrane (typically made of nitrocellulose) due to the assistance of the capillary forces to the detection zone, that consists of another type of antibodies that also specific to molecules of interest to be detected in a sample, but to be specific to the different part (that called epitope) of those molecules. The detection of the analyte of interest on the test line results in an appearance of darker line, while the proper liquid sample flow through the strip test porous membrane results in an appearance of the control line.

Despite recent achievements in area of immunoassay research and development, there is an unmet need for a sensitive and selective analytical method for improving the detection limit.

### SUMMARY OF THE INVENTION

The inventor has surprisingly discovered an enhancement composition that improves analytical sensitivity of detection of an analyte in a liquid sample.

The present invention provides a method for detecting an analyte in a liquid test sample. The method comprises mixing the test sample with a liquid enhancement composition to make a mixture. The enhancement composition comprises one or more non-ionic amphiphilic polymeric compounds selected from the group consisting of hydroxyethylcellulose (HEC), hydroxypropylcellulose (HPC), methylcellulose (MC), hydroxyethyl methyl cellulose (HEMC), and hydroxypropylmethylcellulose (HPMC) and a combination thereof. The mixture comprises the one or more non-ionic amphiphilic polymeric compounds at a combined concentration of 10-1000 times of the critical aggregation concentration (CAC) of the one or more non-ionic amphiphilic polymeric compounds. The mixture has a surface tension σ of 55-67 mN/m. The method also comprises contacting the mixture with a binding agent capable of forming a complex with the analyte. The presence of the complex of analyte and the binding agent indicates the presence of the analyte in the test sample.

The enhancement composition may further comprise an agent selected from the group consisting of bovine serum albumin, D-fructose, gelatin, pectin, sodium bicarbonate and a combination thereof.

The mixture may further comprise bovine serum albumin at 0.1-0.4 mg/ml based on the total volume of the mixture. The mixture may further comprise D-fructose at 0.01-0.7 wt % based on the total weight of the mixture. The mixture may further comprise gelatin at 0.001-0.05 wt % and/or pectin at 0.1-1 wt %, each wt % based on the total weight of the mixture. The mixture may further comprise sodium bicarbonate at 0.01-0.2 wt % based on the total weight of the mixture.

The analyte may be selected from the group consisting of peptides, proteins, polysaccharides, polymers, and nucleic acids (e.g., oligonucleotides, polynucleotides, DNA, RNA, or mRNA). The analyte may be an antibody or an antigen.

The method may further comprise mixing the test sample and the enhancement composition on a mixing material. The mixing material may comprise interstices, channels, cavities and/or pores.

The present invention also provides a mixing device. The mixing device comprises a first compartment comprising a liquid test sample; a second compartment comprising a liquid enhancement composition, which enhancement composition comprises one or more non-ionic amphiphilic polymeric compounds selected from the group consisting of hydroxyethylcellulose (HEC), hydroxypropylcellulose (HPC), methylcellulose (MC), hydroxyethyl methyl cellulose (HEMC), and hydroxypropylmethylcellulose (HPMC) and a combination thereof; and a third compartment in which the test sample from the first compartment and the enhancement composition from the second compartment are mixed to make a mixture, wherein the mixture comprises the one or more non-ionic amphiphilic polymeric compounds at a combined concentration of 10-1000 times of the critical aggregation concentration (CAC) of the one or more non-ionic amphiphilic polymeric compounds, and the mixture has a surface tension σ of 55-67 mN/m. The liquid test sample may comprise an analyte selected from the group consisting of peptides, proteins, polysaccharides, polymers, nucleic acids. The analyte may be an antibody or an antigen.

The mixing device may be selected from the group consisting of pipette, syringe, transfer plastic pipette, pipette droppers, eye dropper, pasteur pipette, disposable pipettes, capillaries and microfluidics.

The third compartment may comprise a mixing material, and the test sample and the enhancement composition may be mixed on the mixing material. The mixing material may comprise interstices, channels, cavities and/or pores.

### BRIEF DESCRIPTION OF THE DRAWINGS

FIGS. 1A-B show results of a saturation binding experiment. A: Commercial LFA curve shows binding of a urine sample spiked with hCG at different concentrations to the commercially available lateral flow rapid test strip without any modification. Modified LFA curve shows binding of the hCG sample to the same model of the commercially available lateral flow rapid test strip modified according to the present invention description. An average data of 3 experiments were used to plot these curves. B: Side-by-side comparison of 3 LFA test strips after injection in each of them 50 microliters of one of 3 types of urine solution: Control 0 mU/ml sample-urine itself with no hCG added; Commercial-25 mU/ml sample-urine spiked with 25 mU/ml hCG; Modified-25 mU/ml sample-urine spiked with 25 mU/ml hCG in modified according to the present invention approach. Test strips have control line and test line.

FIG. 2 shows time dependence of the LFA test line for 25 mU/ml hCG in normal human urine. Triangles—control (no hCG), squares—commercial test, dots—modified test. The comparison of the test line intensities of 3 LFA tests at different moments of time after injection in each of them 50 microliters of one of 3 types of urine solution: Control 0 mU/ml sample—urine itself with no hCG added; Commercial 25 mU/ml sample—urine spiked with 25 mU/ml hCG; Modified 25 mU/ml sample—urine spiked with 25 mU/ml hCG in modified according to the present invention approach.

FIG. 3 shows time dependence of the LFA test line for 5 mU/ml hCG in normal human urine. Triangles—control (no hCG), squares—commercial test, dots—modified test. The comparison of the test line intensities of 3 LFA tests at different moments of time after injection in each of them 50 microliters of one of 3 types of urine solution: Control 0 mU/ml sample—urine itself with no hCG added; Commercial 5 mU/ml sample—urine spiked with 5 mU/ml hCG; Modified 5 mU/ml sample—urine spiked with 5 mU/ml hCG in modified according to the present invention approach.

FIGS. 4A-B show side-by-side comparison of 3 LFA test strips after injection in each of them 50 microliters of one of 3 types of urine solutions: Control 0 mU/ml sample—urine itself with no hCG added; Commercial 25 mU/ml sample—urine spiked with 25 mU/ml hCG; Modified 25 mU/ml sample—urine spiked with 25 mU/ml hCG in modified according to the present invention approach. A: Exhibit A. B: Exhibit B. Exhibits A and B are referring to 2 groups of LFA test strips obtained from 2 different commercial companies. Test strips A have control line, reference line and test line. Test strips B have control line and test line.

FIG. 5 shows results of a COVID-19 IgM positive serum sample in a saturation binding experiment with commercial and modified tests.

FIGS. 6A-B show side-by-side comparison of LFA test strips. A: Side-by-side comparison of LFA test strips after injection in each of them 50 microliters of 1:25 dilution of standard COVID 19 IgM positive serum control sample: Control sample—negative serum control sample; Commercial 1:25 dilution samples—1:25 dilution of standard COVID 19 IgM positive serum control sample; Modified 1:25 dilution samples—1:25 dilution of standard COVID 19 IgM positive serum control sample in modified approach according to the present invention. Commercial LFA test strips are the commercially available lateral flow rapid test strips without any modification, were used according to the company manual protocol. Improved LFA test strips are the same model of the commercially available lateral flow rapid test strips additionally modified according to the present invention. Photos of the LFA test strips were taken at about 10 minutes of analysis time. B: Side-by-side comparison of LFA test strips after injection in each of them 50 microliters by the same procedure as described in A, the dilutions shown cover the range from 1:10 to 1:333, namely: 1:10, 1:25, 1:50, 1:100, or 3:1000.

FIG. 7 shows enhancement transfer-mixing devices. Enhancement transfer-mixing device (single-channel syringe A-1 or transfer pipette with flexible compartment A-2) filled with mixing material (1) soaked with a specific amount of an enhancement composition solution/mixture, 2—plunger, 3—barrel, 4—cup. B and C—sucking a sample; D and E—delivering a sample to LFA (6) sample pad (8). 7—LFA absorption pad.

### DETAILED DESCRIPTION OF THE INVENTION

The present invention relates to a method for detecting an analyte in a sample and a mixing device useful for the detection. The invention is based on the inventor's surprising discovery that a liquid enhancement composition comprising non-ionic amphiphilic polymeric compounds, for example, hydroxyethylcellulose (HEC), hydroxypropylcellulose (HPC), methylcellulose (MC), hydroxyethyl methyl cellulose (HEMC), and hydroxypropylmethylcellulose (HPMC), at a low concentration enhances binding properties of molecules in a solution having a surface tension of, for example, 55-67 mN/m. In particular, the use of these compounds at a concentration of 10-1000 times of their critical aggregation concentration (CAC) enhance analytical sensitivity and/or analytical selectivity of detection of an analyte in a liquid sample.

The term “analytical sensitivity” as used herein refers to the smallest amount of substance in a sample that can be measured accurately by an assay.

The term “analytical selectivity” as used herein refers to the ability of an assay to measure molecules of interest among other molecules in a sample.

A method for detecting an analyte in a liquid test sample is provided. The method comprises mixing the test sample with an enhancement composition to make a mixture. The enhancement composition is a liquid and comprises one or more non-ionic amphiphilic polymeric compounds. The method further comprises contacting the mixture with a binding agent. The binding agent is capable of forming a complex with the analyte. The presence of the complex of analyte and the binding agent indicates the presence of the analyte in the test sample.

According to the detection method, the non-ionic amphiphilic polymeric compounds may be selected from the group consisting of hydroxyethylcellulose (HEC), hydroxypropylcellulose (HPC), methylcellulose (MC), hydroxyethyl methyl cellulose (HEMC), and hydroxypropylmethylcellulose (HPMC) and a combination thereof.

According to the detection method, the mixture may comprise the one or more non-ionic amphiphilic polymeric compounds at a combined concentration of 10-1000, 10-500 or 50-200 times of the critical aggregation concentration (CAC) of the one or more non-ionic amphiphilic polymeric compounds. The mixture may have a surface tension σ of about 10-100 mN/m, 50-100 mN/m, 50-70 mN/m, 55-67 mN/m or 60-65 mN/m.

According to the detection method, the analyte may be selected from the group consisting of peptides, proteins, polysaccharides, polymers, and nucleic acids. Examples of the nucleic acids include oligonucleotides, polynucleotides, DNA, RNA, mRNA, and combinations thereof. The analyte may be an antibody or an antigen.

The liquid test sample suitable for the detection method may be a biological sample. Examples of the liquid test sample include a bodily fluid, a breath vapor, a gas, a medium, or a combination thereof. The bodily fluid may be selected from the group consisting of whole blood, amniotic fluid, aqueous humour and vitreous humour, bile, blood serum, breast milk, cerebrospinal fluid, chyle, chyme, endolymph, perilymph, exudates, feces (diarrhea), female ejaculate, gastric acid, gastric juice, lymph, mucus (e.g., nasal drainage and phlegm), pericardial fluid, peritoneal fluid, pleural fluid, pus, rheum, saliva, sebum (e.g., skin oil), serous fluid, semen, smegma, sputum, synovial fluid, sweat, tears, urine, vaginal secretion, and vomit. The liquid test sample may comprise bacteria or viruses.

According to the detection method, the enhancement composition may further comprise an agent. The agent may be a water-soluble polymeric carbohydrate compound. The agent may be selected from the group consisting of bovine serum albumin, D-fructose, gelatin, pectin, sodium bicarbonate, alginate, cellulose, chitosan, chondroitin sulfate, dextran, heparin, xylan, hydroxypropyl methylcellulose, hydroxypropyl cellulose, methyl cellulose, carboxymethyl cellulose, and a combination thereof. The mixture may further comprise bovine serum albumin at about 0.1-0.4 mg/ml based on the total volume of the mixture; D-fructose at about 0.01-0.7 wt % based on the total weight of the mixture; gelatin at about 0.001-0.05 wt % each wt % based on the total weight of the mixture; pectin at about 0.1-3 wt %, each wt % based on the total weight of the mixture; gelatin at about 0.001-0.05 wt % and/or pectin at about 0.1-1 wt %, each wt % based on the total weight of the mixture; sodium bicarbonate at about 0.01-0.2 wt % based on the total weight of the mixture; alginate at about 0.05-0.5 wt % based on the total volume of the mixture; cellulose at about 0.05-0.5 wt % based on the total volume of the mixture; chitosan at about 0.05-0.5 wt % based on the total volume of the mixture; chondroitin sulfate at about 0.05-0.5% wt % based on the total volume of the mixture; dextran at 0.05-1.5 wt % based on the total volume of the mixture; heparin at about 0.05-1.5% wt % based on the total volume of the mixture; xylan at about 0.05-1.5% wt % based on the total volume of the mixture; hydroxypropyl methylcellulose at about 0.05-0.15% wt % based on the total volume of the mixture; hydroxypropyl cellulose at about 0.05-0.15% wt % based on the total volume of the mixture; methyl cellulose at about 0.05-0.15% wt % based on the total volume of the mixture; and/or carboxymethyl cellulose at about 0.05-wt % based on the total volume of the mixture. The agent may be a bacteriostatic preservative such as sodium azide.

The detection method may further comprise mixing the test sample and the enhancement composition on a mixing material. The mixing material may be capable of absorbing a water-based solution. The mixing material may be soft or hard. The mixing material may comprise interstices, channels, cavities and/or pores. The pores may be cylindrical, funnel, and bottle shaped. The mixing material may be selected from the group consisting of filter paper, sand, particles, microparticles, agarose beads, hydrogels, woven and non-woven fabric, sponge, wood fiber, bandage, cloth, dry or impregnated gauze, plastic films, gels, foams, hydrocolloids, alginates, hydrogels, polysaccharide pastes, granules, beads, woven or non-woven fibers of cotton, rayon, polyester, ceramic filters, three-dimensional networks composed of a cross-linked polymers, threads and fabric materials. The mixing material may be porous with various pore sizes (e.g., micro-, meso-, and macropores), with various arrangements (e.g., inter-aggregation and inter-cluster), with various shapes (e.g., cylinders, cone, slit, and sphere), and with different water-based solution-accessibility (e.g., open and close pores). The mixing material may be soaked with or filled with a dried component of the enhancement composition.

A mixing device is also provided. The mixing device comprises three components. The first compartment comprises a liquid test sample. The second compartment comprises an enhancement composition. The enhancement composition is a liquid and comprises one or more non-ionic amphiphilic polymeric compounds. In the third compartment, the test sample from the first compartment and the enhancement composition from the second compartment are mixed to make a mixture.

The mixing device may be selected from the group consisting of pipette, syringe, transfer plastic pipette, pipette droppers, eye dropper, pasteur pipette, disposable pipettes, capillaries and microfluidics.

Each of the first, second and third compartments may be of any shape, for example, cube, cylinder, sphere, cone, or slit. Each of the first, second and third compartments may include a separate section of a structure or container in which certain items are kept separately from others.

In the mixing device, the non-ionic amphiphilic polymeric compounds in the second compartment may be selected from the group consisting of hydroxyethylcellulose (HEC), hydroxypropylcellulose (HPC), methylcellulose (MC), hydroxyethyl methyl cellulose (HEMC), and hydroxypropylmethylcellulose (HPMC) and a combination thereof.

The mixture in the third compartment may comprise the one or more non-ionic amphiphilic polymeric compounds at a combined concentration of 10-1000, 10-500 or 50-200 times of the critical aggregation concentration (CAC) of the one or more non-ionic amphiphilic polymeric compounds. The mixture may have a surface tension σ of about 10-100 mN/m, 50-100 mN/m, 50-70 mN/m, 55-67 mN/m or 60-65 mN/m.

In the mixing device, the enhancement composition may further comprise an agent. The agent may be a water-soluble polymeric carbohydrate compound. The agent may be selected from the group consisting of bovine serum albumin, D-fructose, gelatin, pectin, sodium bicarbonate, alginate, cellulose, chitosan, chondroitin sulfate, dextran, heparin, xylan, hydroxypropyl methylcellulose, hydroxypropyl cellulose, methyl cellulose, carboxymethyl cellulose, and a combination thereof. The mixture may further comprise bovine serum albumin at about 0.1-0.4 mg/ml based on the total volume of the mixture; D-fructose at about 0.01-0.7 wt % based on the total weight of the mixture; gelatin at about 0.001-0.05 wt % each wt % based on the total weight of the mixture; pectin at about 0.1-3 wt %, each wt % based on the total weight of the mixture; gelatin at about 0.001-0.05 wt % and/or pectin at about 0.1-1 wt %, each wt % based on the total weight of the mixture; sodium bicarbonate at about 0.01-0.2 wt % based on the total weight of the mixture; alginate at about 0.05-0.5 wt % based on the total volume of the mixture; cellulose at about 0.05-0.5 wt % based on the total volume of the mixture; chitosan at about 0.05-0.5 wt % based on the total volume of the mixture; chondroitin sulfate at about 0.05-0.5% wt % based on the total volume of the mixture; dextran at 0.05-1.5 wt % based on the total volume of the mixture; heparin at about 0.05-1.5% wt % based on the total volume of the mixture; xylan at about 0.05-1.5% wt % based on the total volume of the mixture; hydroxypropyl methylcellulose at about 0.05-0.15% wt % based on the total volume of the mixture; hydroxypropyl cellulose at about 0.05-0.15% wt % based on the total volume of the mixture; methyl cellulose at about 0.05-0.15% wt % based on the total volume of the mixture; and/or carboxymethyl cellulose at about 0.05-wt % based on the total volume of the mixture. The agent may be a bacteriostatic preservative such as sodium azide.

The analyte in the mixing device may be selected from the group consisting of peptides, proteins, polysaccharides, polymers, and nucleic acids. Examples of the nucleic acids include oligonucleotides, polynucleotides, DNA, RNA, mRNA, and combinations thereof. The analyte may be an antibody or an antigen.

In the third compartment, the test sample and the enhancement composition may be mixed on a mixing material. The mixing material may be capable of absorbing a water-based solution. The mixing material may be soft or hard. The mixing material may comprise interstices, channels, cavities and/or pores. The pores may be cylindrical, funnel, and bottle shaped. The mixing material may be selected from the group consisting of filter paper, sand, particles, microparticles, agarose beads, hydrogels, woven and non-woven fabric, sponge, wood fiber, bandage, cloth, dry or impregnated gauze, plastic films, gels, foams, hydrocolloids, alginates, hydrogels, polysaccharide pastes, granules, beads, woven or non-woven fibres of cotton, rayon, polyester, ceramic filters, three-dimensional networks composed of a cross-linked polymers, threads and fabric materials. The mixing material may be porous with various pore sizes (e.g., micro-, meso-, and macropores), with various arrangements (e.g., inter-aggregation and inter-cluster), with various shapes (e.g., cylinders, cone, slit, and sphere), and with different water-based solution-accessibility (e.g., open and close pores). The mixing material may be soaked with or filled with a dried component of the enhancement composition.

In the mixing device, the liquid test sample may be a biological sample. Examples of the liquid test sample include a bodily fluid, a breath vapor, a gas, a medium, and a combination thereof. The bodily fluid may be selected from the group consisting whole blood, amniotic fluid, aqueous humour and vitreous humour, bile, blood serum, breast milk, cerebrospinal fluid, chyle, chyme, endolymph, perilymph, exudates, feces (diarrhea), female ejaculate, gastric acid, gastric juice, lymph, mucus (e.g., nasal drainage and phlegm), pericardial fluid, peritoneal fluid, pleural fluid, pus, rheum, saliva, sebum (e.g., skin oil), serous fluid, semen, smegma, sputum, synovial fluid, sweat, tears, urine, vaginal secretion, and vomit. The liquid test sample may comprise bacteria or viruses.

The concentration of an analyte may be determined in any analytical assay known in the art. Examples of the analytical assays include immunoassays, radioimmunoassays (RIA), counting immunoassays (CIA), enzyme immunoassays (EIA), enzyme-linked immunosorbent assays (ELISA), fluoro-immunoassays (FIA), chemiluminescence immunoassays (CLIA), magneto-actuated immunoassays, agglutination immunoassays, competitive immunoassays, non-competitive immunoassays, ELISA-type of assays, competitive assays, direct sandwiches and indirect sandwiches.

The present invention provides a detection method with improved analytical sensitivity. The analytical sensitivity of the method for detecting an analyte in a liquid sample, for example, a biological liquid sample, may be improved by, for example, at least about 5%, 10%, 20%, 30%, 40%, 50%, 60%, 70%, 80%, 90%, 100%, 200%, 300%, 400%, 500% or 1000%, or about 5-50%, 5-100%, 5-500%, 5-1000%, 50-100%, 50-500%, 50-1000%, 100-500%, 100-1000% or 500-1000%, as compared with that of the same detection method except without the enhancement composition.

The present invention provides a detection method with improved selectivity. The selectivity of the method for detecting an analyte in a liquid sample, for example, a biological liquid sample, may be improved by, for example, at least about 5%, 10%, 20%, 30%, 40%, 50%, 60%, 70%, 80%, 90%, 100%, 200%, 300%, 400%, 500% or 1000%, or about 5-50%, 5-100%, 5-500%, 5-1000%, 50-100%, 50-500%, 50-1000%, 100-500%, 100-1000% or 500-1000%, as compared with that of the same detection method except without the enhancement composition.

According to the present invention, the improved analytical sensitivity of detection of an analyte in a biological liquid sample may be achieved by mixing at, for example, the biological liquid sample of interest (e.g., blood, sweat, urine, saliva, tears), which may be used as is or diluted with a sample diluent if needed, with one or more of non-ionic amphiphilic polymeric compounds, also referred herein as “enhancement compounds,” including but not limiting to non-ionic cellulose ethers, for example, hydroxyethylcellulose (HEC), hydroxypropylcellulose (HPC), methylcellulose (MC), hydroxyethyl methyl cellulose (HEMC), and hydroxypropylmethylcellulose (HPMC) or a mixture thereof at various proportions, and optionally with an agent, also referred herein as “other enhancement compound.” Examples of the agent include, but not limited to, bovine serum albumin at, for example, 0.1-0.4 mg/ml; D-fructose at, for example, 0.01-0.7% (w/v); gelatin at, for example, 0.001-0.05% (w/v); pectin at, for example, 0.1-1% (w/v); sodium bicarbonate at, for example, 0.01-0.2% (w/v).

The detection method of the present invention may be a lateral flow assays (LFA), particle-based assay or immuno-assay. The improvement or enhancement of detection may be observed in an equilibrium dissociation constant Kd, an apparent dissociation constant and/or a ratio of the rate constants (i.e., dissociation rate constant koff to association rate constant kon), which reflects interaction between molecules in the assay, for example, the interaction between an antibody and an antigen. Examples of suitable molecules include peptides, proteins, polysaccharides, polymers, and nucleic acids (e.g., oligonucleotides, polynucleotides, DNA, RNA, mRNA). For example, the visibility (i.e., contrast) of a LFA test line may be improved.

The non-ionic amphiphilic polymeric compounds may be present in the mixture according to the present invention at a concentration determined by multiplication of the critical aggregation concentration (CAC) of the compounds by a specific factor, for example, by adjusting the surface tension of the final solution to a specific range of values. The agent may be used for adjustment of the concentration of the non-ionic amphiphilic polymeric compounds and/or the surface tension of the final solution.

The enhancement composition may be prepared by mixing the enhancement compounds and the agent. The enhancement compounds are non-ionic amphiphilic polymeric compounds, including non-ionic cellulose ethers (e.g., hydroxyethylcellulose (HEC), hydroxypropylcellulose (HPC), methylcellulose (MC), hydroxyethylmethylcellulose (HEMC), and hydroxypropylmethylcellulose (HPMC) or mixture thereof at various proportions). For example, the enhancement composition may be Methocel™ K100LV Hydroxypropyl Methylcellulose (HPMC) containing about 19.0-24.0 wt % Methoxyl and about 7.0-12.0% of Hydroxypropoxyl.

### Embodiments

1) Mixing a biological liquid sample of interest (e.g., blood, sweat, urine, saliva, tears), which may be used as is or diluted with a sample diluent if needed, under specified condition with an enhancement composition comprising one or more of the non-ionic amphiphilic polymeric compounds, including but not limiting to non-ionic cellulose ethers (e.g., hydroxyethylcellulose (HEC), hydroxypropylcellulose (HPC), methylcellulose (MC), hydroxyethylmethylcellulose (HEMC), and hydroxypropylmethylcellulose (HPMC) or mixture thereof) a specified proportions, and optionally an agent.

2) The non-ionic amphiphilic polymeric compounds may be present at a specified concentration determined by multiplication of the critical aggregation concentration (CAC) of the compounds by a specific factor such that the combined concentration of the non-ionic amphiphilic polymeric compounds in the mixture is about 10-500 or 50-200 times of the CAC.

3) The concentrations of the non-ionic amphiphilic polymeric compounds and an optional agent in the enhancement composition may be used to adjust the surface tension σ of the resulting solution to be in a range of about 55-67 or 60-65 mN/m.

4) The agent may be used to adjust parameters of the non-ionic amphiphilic polymeric compounds if needed such as 1) concentration determined by multiplication of the critical aggregation concentration (CAC) of the non-ionic amphiphilic polymeric compounds by a specific factor and 2) surface tension of the final solution to the specific range of values.

5) A mixing device filled with an enhancement composition.

6) Examples of the mixing device include but not limited to: pipette, syringe, transfer plastic pipette, pipette droppers, eye dropper, pasteur pipette, disposable pipettes, capillaries, microfluidics, and other alike.

7) A mixing device comprising a mixing compartment, which may be filled with a mixing material.

8) Examples of the mixing compartment include any kind of confined space or compartment of any shape such as cube, cylinder, sphere, cone, slit, other shape and other alike.

9) The mixing device may include any kind of a separate section of a structure or container in which certain items can be kept separate from others.

10) The mixing material may be capable of absorbing or containing water-based solutions.

11) The mixing material may be soft or hard.

12) The mixing material may include a series of interstices, channels, and cavities and other pores.

13) The pores in the mixing material may be of various shapes, including but not limited to: cylindrical, funnel, and bottle-shaped.

14) The mixing material may include various porous materials with various pore sizes (e.g., micro-, meso-, and macropores), with various arrangements (e.g., inter-aggregation and inter-cluster), in various shapes (e.g., cylinders, cone, slit, sphere, and other alike), and with different water-based solution-accessibility (e.g., open and close pores).

15) Examples of the mixing material may include but not limited to: filter paper, sand, particles, microparticles, agarose beads, hydrogels, woven and non-woven fabric, sponge, wood fiber, bandage, cloth, dry or impregnated gauze, plastic films, gels, foams, hydrocolloids, alginates, hydrogels, and polysaccharide pastes, granules and beads, woven or non-woven fibres of cotton, rayon, and polyester, ceramic filters, three-dimensional networks composed of a cross-linked polymers, threads, fabric materials, any other porous material, and other alike.

16) The mixing material may be soaked with or filled with a dried component of the enhancement composition.

### Example 1. Saturation Binding Experiment

In a saturation binding experiment, the concentration of a ligand was varied and the binding properties of the ligand was measured. The goal was to determine the constant K_{D} (i.e., ligand concentration that binds to half the receptor sites at equilibrium) and Bₘₐₓ (i.e., maximum number of binding sites).

Bₘₐₓ is the maximum specific binding in the same units as Y. It is the specific binding extrapolated to very high concentrations of ligand, and so its value is almost always higher than any specific binding measured in the experiment. K_{D} is the equilibrium dissociation constant, a ratio of k_{off}/kₒₙ, between the antibody and its antigen. K_{D} and affinity are inversely related. K_{D} is in the same units as X. It is the ligand concentration needed to achieve a half-maximum binding at equilibrium. Therefore, the lower the K_{D} value and thus the higher the affinity of the antibody.

The ratio between equilibrium dissociation constants K_{D-com} (commercial LFA test) and K_{D-mod} (improved by the present invention LFA test) were determined from the binding curve fitting by utilizing the model for specific binding: Y=Bₘₐₓ*X/(K_{D}+X)

Other methods and equations of determination of the equilibrium dissociation constants are also possible.

In one of the embodiments, related to detection of the hCG (Human Chorionic Gonadotropin) by LFA method, the improvement for the equilibrium dissociation constants K_{D} was determined about 2.4 times for the hCG containing samples (K_{D-com}=1762±234 mU/ml and K_{D-mod}=745.7±241 mU/ml), additionally to the significant improvement for visibility (contrast) of the Test line (FIGS. 1, 2, 3, 5).

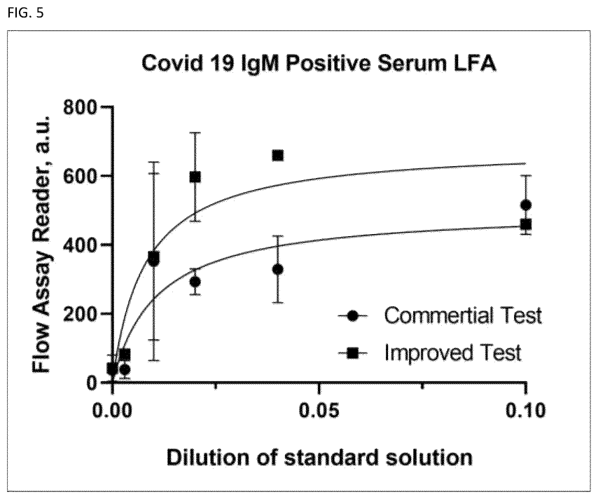

In one of the embodiments (FIG. 4), it was demonstrated about 2-5 times better LFA analytical sensitivity in comparison to the commercially available Tests, such as: OSOM hCG Combo (Sekisui Diagnostics); 2) Adexus, Dx hCG; 3) Clinitest hCG (Siemens); 4) Clarity Diagnostics hCG; 5) Clinical Guard hCG.

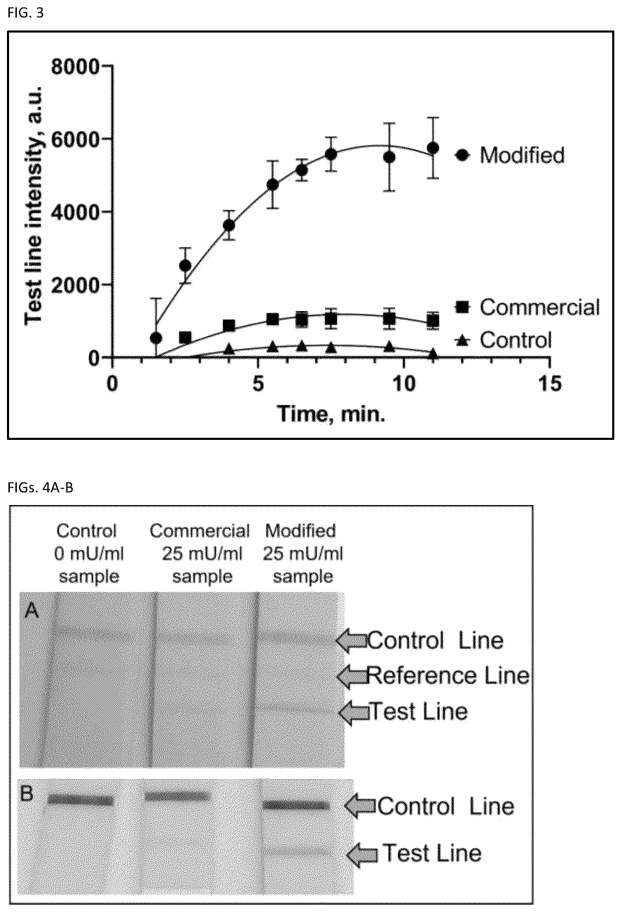

Besides from the better analytical sensitivity, a modified pregnancy LFA according to the present invention demonstrates much better “visibility” of the test line (FIGS. 1B, 2, 3, 4), including condition at low hCG concentration (˜5 mU/ml) (FIG. 3). Comparison was done in a human urine spiked with hCG. Test Prototype has been developed and verified in comparison with the commercially available Tests.

A commercially available LFA test was used according to manufacturer manual and the modified LFA test was used according to the present invention. Results are presented in FIGS. 1, 2 and 3.

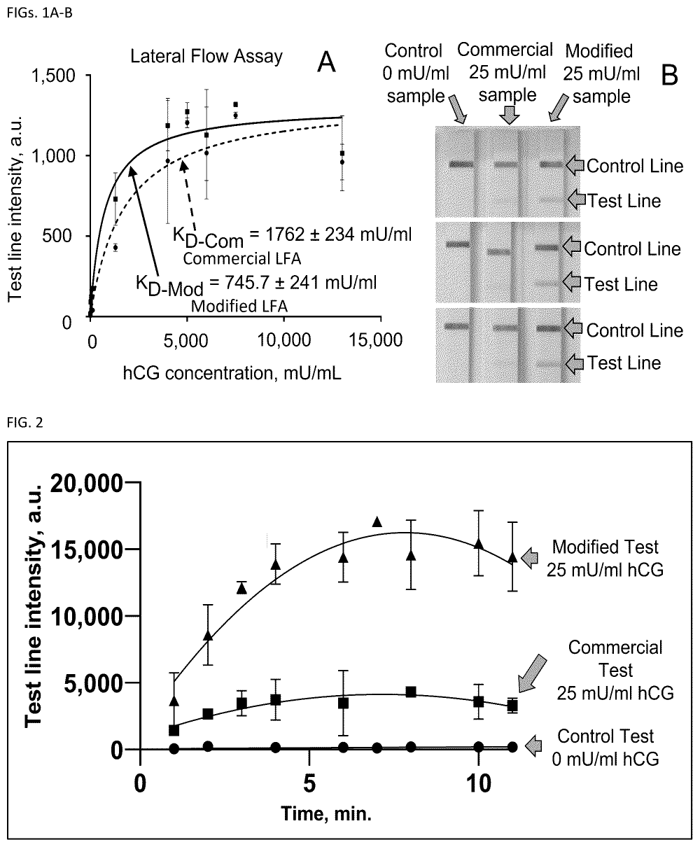

FIG. 2 shows the time dependence of the LFA test line for 25 mU/ml hCG in normal human urine. Triangles represent control (no hCG), squares-commercial test, dots-modified test. The comparison of the test line intensities of 3 LFA tests at different moments of time were done after injection in each of them 50 microliters of one of 3 types of urine solution: “Control 0 mU/ml sample” represents urine itself with no hCG added; “Commercial 25 mU/ml sample”—urine spiked with 25 mU/ml hCG; “Modified 25 mU/ml sample”—urine spiked with 25 mU/ml hCG in modified according to the present invention approach. As shown in FIG. 2, the intensity of the modified test for the detection of 25 mU/ml hCG in human urine is about 15,000 a.u. and for the commercial test is about 4,000 a.u. with about 3.8 times improvement (FIG. 2), and for the detection of 5 mU/ml hCG in human urine it is about 6,000 a.u. and for the commercial test is about 1,000 a.u. with about 6 times improvement (FIG. 3).

In one of the embodiments, it was done the side-by-side comparison of 3 LFA test strips after injection in each of them 50 microliters of one of 3 types of urine solutions (FIG. 1): Control 0 mU/ml sample—urine itself with no hCG added; Commercial 25 mU/ml sample—urine spiked with 25 mU/ml hCG; Modified 25 mU/ml sample—urine spiked with 25 mU/ml hCG in modified according to the present invention approach (see text for more details). FIG. 1A—Exhibit A and FIG. 1B—Exhibit B are referring to 2 groups of LFA test strips obtained from 2 different commercial companies. Test strips A have control line, reference line and test line. Test strips B have control line and test line.

In one of the embodiments, a saturation binding experiment was performed with COVID 19 IgM positive plasma control samples provided by the RayBioteck company (GA) (FIG. 5). The purpose of the experiment was to determine the visibility (contrast) of the Test line for purpose of improvement of the following outcomes of the lateral flow assays (LFA): the test line visibility and the binding property of the COVID 19 IgM antibody with a relevant COVID 19 antigen. We used the test strips reader ESEQuant LR3 lateral flow reader (DIALUNOX GmbH, Stockach, Germany) to measure the intensity of the test and control lines of the LFA test.

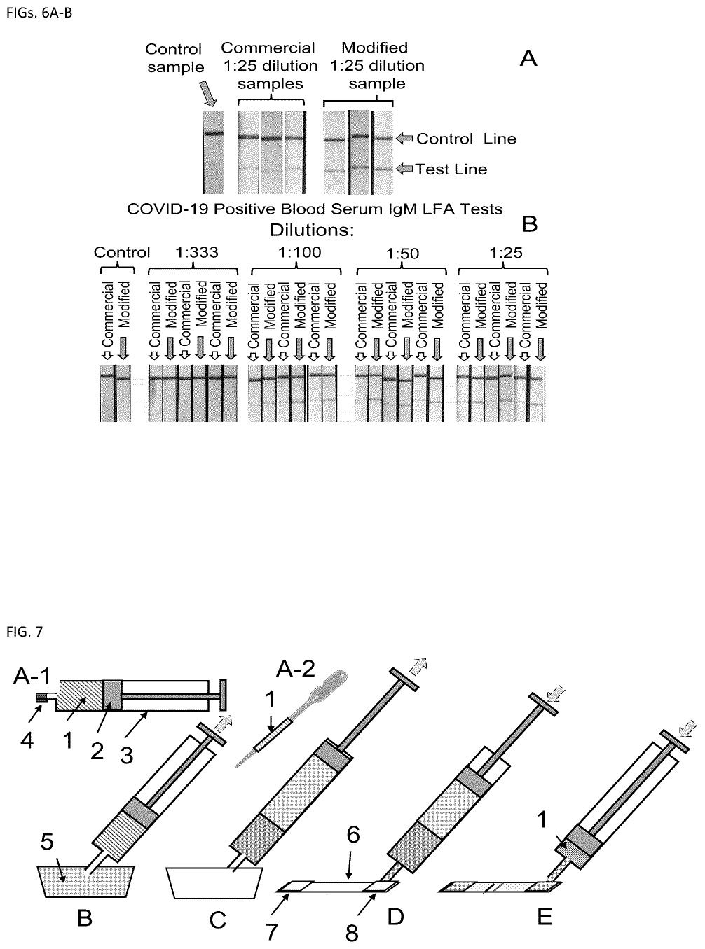

Improvement of equilibrium dissociation constants Kd was determined about 1.7 times for the COVID 19 IgM positive serum control samples, additionally to the significant improvement for visibility (contrast) of the Test line (FIG. 5: commercial test strips and modified test strips). Improvement for visibility (contrast) of the LFA test lines was determined in a range from about 10-times at 1:333 IgM COVID-19 positive sample dilution to about 2-times for all other checked IgM COVID-19 positive sample dilutions (namely: 1:10, 1:25, 1:50, 1:100) when comparing the commercial test strips and modified according to the present invention test strips (FIG. 6B).

### Example 2. Detection of Human Chorionic Gonadotrophin (hCG)

A serial dilution was performed with a hCG sample. The dilutions cover the range hCG concentrations from 5 mU/ml to 13,000 mU/ml, for example, 5, 10, 25, 50, 65, 130, 1,300, and 13,000, mIU/ml.

A mixing device, 1 ml disposable syringe, was partially filled with a mixing material, cellulose acetate tow fibers, (FIG. 7). The enhancement composition was an aqueous solution containing:
(1) NaCl at 8-10 g/L, KCl at 0.2-0.4 g/L, Na₂HPO₄ at 1.4-1.5 g/L, KH₂PO₄ at 0.2-0.3 g/L, methyl cellulose at 0.1-0.15% (w/v), and sodium azide at 0.02% (w/v);
(2) NaCl at 0-10 g/L, methyl cellulose at 0.1-0.15% (w/v) and sodium azide at 0.02% (w/v);
(3) NaCl at 8-10 g/L, KCl at 0.2-0.4 g/L, Na₂HPO₄ at 1.4-1.5 g/L, KH₂PO₄ at 0.2-0.3 g/L, methyl cellulose at 0.1-0.15% (w/v), pectin at 0.1-0.05% (w/v), sodium bicarbonate at 0.01-0.2% (w/v), and sodium azide at 0.02% (w/v);
(4) NaCl at 8-10 g/L, KCl at 0.2-0.4 g/L, Na₂HPO₄ at 1.4-1.5 g/L, KH₂PO₄ at 0.2-0.3 g/L, methyl cellulose at 0.1-0.15% (w/v), hydroxypropyl cellulose at 0.05-0.15% (w/v), and sodium azide at 0.02% (w/v);
(5) NaCl at 8-10 g/L, KCl at 0.2-0.4 g/L, Na₂HPO₄ at 1.4-1.5 g/L, KH₂PO₄ at 0.2-0.3 g/L, hydroxypropyl cellulose at 0.05-0.15% (w/v)), and sodium azide at 0.02% (w/v); or
(6) bovine serum albumin at 0.1-0.4 mg/ml, D-fructose at 0.01-0.7% (w/v), gelatin at 0.001-0.05% (w/v), alginate at 0.01-0.15% (w/v)), cellulose at 0.01-0.15% (w/v), chitosan at 0.01-0.15% (w/v)); chondroitin sulfate at 0.05-0.15% (w/v), dextran at 0.05-0.15% (w/v), heparin at 0.01-0.15% (w/v), xylan at 0.05-1.5% (w/v), hydroxypropyl methylcellulose at 0.01-0.15% (w/v), hydroxypropyl cellulose at 0.01-0.15% (w/v), methyl cellulose at 0.01-0.15% (w/v), carboxymethyl cellulose at 0.01-0.15% (w/v), pectin at 0.1-3% (w/v), sodium bicarbonate at 0.01-0.2% (w/v), NaCl at 8-10 g/L, KCl at 0.2-0.4 g/L, Na₂HPO₄ at 1.4-1.5 g/L, KH₂PO₄ at 0.2-0.3 g/L, and sodium azide at 0.02% (w/v).

The hCG samples were directly mixed in a container with the enhancement composition in an amount of 10 μl to 50 μl per 1 ml of the sample. The resulting mixture remained as an aqueous solution containing the same ingredients in the enhancement composition at essentially the same concentrations in the mixture.

The surface tension of the mixture was in the range from 55 to 67 mN/m or 60 to 65 mN/m. The surface tension was adjusted if needed by changing the amount of the mentioned above compounds.

The mixing device with the mixing material and the enhancement composition (FIG. 7) was used to deliver the mentioned above dilutions of sample to a LFA test strip by sucking in and releasing the specific amount of liquid sample, for example, according to LFA recommended sample volume, containing of one of the mentioned above hCG at the specified concentrations, i.e., 5, 10, 25, 50, 65, 130, 1300, 13000 mU/ml.

About 0.5-1 mL of a liquid sample containing of one of the mentioned above hCG in specified above concentrations was released into a small clear jar (or cup). The LFA test strip was immersed in liquid sample for the amount of time indicated in the LFA instructions, for example, 5-8 minutes. After that, LFA test strip was placed on a holder inside of the ESEQuant LR3 lateral flow reader (DIALUNOX GmbH, Stockach, Germany) for reading the test results by scanning the test strip.

For purpose of simultaneous analysis of multiple samples, the immersed test strips were combined into sets by 5 test strips in each set and were placed in the strip test holder for the following analysis as it described above.

Alternatively, about 90-100 μL of a liquid sample containing of one of the mentioned above hCG in specified above concentrations was released into one of the wells in 96-well plate. After 5 wells were filled with a sample solution, a multichannel pipettor was used for simultaneous deposition of about 50 μl of the sample dilution on the sample pad of each of 5 test strips. After that, 5 test stripes were placed on a holder inside of the ESEQuant LR3 (DIALUNOX GmbH, Stockach, Germany) for reading the test results by scanning the test strips preferably every minute.

LFA test strips filled with liquid samples may be taken away from lateral flow reader at a specified moment of time, for example, at the end of the testing time, after 5-8 minutes, or as specified by the LFA test instruction, for visual inspection or for taking the photographic pictures.

The analysis of the photographic pictures was performed by utilizing an image-analyzing software, for example, freely available ImageJ software.

In one of the embodiments, the samples were prepared by spiking a PBS buffer with a specific amount of hCG to reach a hCG concentrations in the range from 5 mU/ml to 13,000 mU/ml, for example, at 5, 10, 25, 50, 65, 130, 1,300, 13,000, mU/ml.

In another embodiment, there samples were prepared by spiking a healthy human urine with a specific amount of hCG to reach a hCG concentrations in the range from 5 mU/ml to 13,000 mU/ml, for example, at 5, 10, 25, 50, 65, 130, 1,300, 13,000, mU/ml.

In yet another embodiment, the software Prism 8 for Windows 64-bit Version 8.4.3 (686) obtained from GraphPad Software, LLC (San Diego, CA) was used for fitting the experimental data points (FIGS. 1-3) and to determine the equilibrium constants (FIG. 1).

### Example 3. Detection of Toxins

Similar to the approach in Example 2, some toxins produced by bacteria (E. coli human pathogens) were detected.

Among the E. coli human pathogens, Shiga toxin-producing strains of E. coli (EHEC) have gained in importance in recent years. The group of EHEC, with their highly pathogenic serovars and other strains are of particular concern. Production of Shiga toxins is the most common criteria for the detection of this group of bacteria. Shiga toxins can be classified into two main categories: Shiga toxin 1 (ST1) and Shiga toxin 2 (ST2). EHEC strains may produce ST1 or ST2 only or both ST1 and ST2 simultaneously. EHEC are capable of initiating life-threatening illnesses, particularly in young children, the elderly or patients with immune deficiency. The main sources of infection are contaminated, raw or insufficiently heated foods of animal origin, e.g., meat and dairy products. The reservoirs for EHEC are cattle, sheep and goats and is spread through their feces. These microorganisms can enter food during the processing of meat and dairy products if hygienic conditions are inadequate. The incidence of food infection caused by Shiga toxin-producing E. coli demands reliable and rapid methods of detection. In addition to traditional culture methods, immunological techniques are becoming more useful due to their improved specificity and analytical sensitivity. We used a commercially available test, ImmunoCard STAT!™ EHEC, an immunological diagnostic test based on the immunochromatographic lateral flow principle, according to manufacturer manual and the LFA test version modified according to the present invention.

A mixing device, a mixing material and an enhancement composition were prepared in a way similar to the protocol in Example 2 (FIG. 7).

A mixing device, 1 ml disposable syringe, was partially filled with a mixing material, cellulose acetate tow fibers, (FIG. 7), which was soaked with an enhancement composition in an amount of 10 μl to 50 μl per 1 ml of a sample to make a mixture. The enhancement composition is an aqueous solution containing NaCl at 8-10 g/L, KCl at 0.2-0.4 g/L, Na₂HPO₄ at 1.4-1.5 g/L, KH₂PO₄ at 0.2-0.3 g/L, methyl cellulose at 0.1-0.15% (w/v) or 0.2-0.25% (w/v), and sodium azide at 0.02% (w/v).

The surface tension of the mixture was in the range from 55 to 67 mN/m or 60 to 65 mN/m. The surface tension was adjusted if needed by changing the amount of the mentioned above compounds.

The mixing device with the mixture material and the enhancement composition (FIG. 7) was used to deliver 1:4 and 1:33 positive control sample (Positive Control reagent) dilutions of the sample to the LFA test strip by sucking in and releasing about 165 μL of the liquid sample on the sample pad of a related test strip. The Positive Control reagent contained formalin-treated (inactivated) shiga toxins ST1 and ST2.

After that, the LFA test strip was placed on a holder inside of the ESEQuant LR3 lateral flow reader (DIALUNOX GmbH, Stockach, Germany) for reading the test results by scanning the test strip preferably every 1 minute.

The LFA test strips filled with liquid samples were taken away from lateral flow reader at the specified moment of time, for example, at the end of the testing time or after about 21 minutes as specified by the LFA test instruction, for visual inspection and for taking the photographic pictures.

Improvement for visibility (contrast) of the LFA test lines related to both shiga toxins ST1 and ST2 was determined to be in a range from 30% to 42% by comparing the commercial test strips with modified according to the present invention test strips (FIG. 4).

### Example 4. Detection of COVID-19 IgM

A saturation binding experiment was done with COVID-19 IgM positive serum control samples provided by the RayBioteck company (GA) (FIG. 5).

A mixing device, 1 ml disposable syringe, was partially filled with a mixing material, cellulose acetate tow fibers, (FIG. 7). The mixing material was soaked with an enhancement composition in an amount of 10 μl to 50 μl per 1 ml of a sample to make a mixture. The enhancement composition was an aqueous solution containing NaCl at 8-10 g/L, KCl at 0.2-0.4 g/L, Na₂HPO₄ at 1.4-1.5 g/L, KH₂PO₄ at 0.2-0.3 g/L, methyl cellulose at 0.1-0.15% (w/v), and sodium azide at 0.02% (w/v). The surface tension of the mixture was in the range from 55 to 67 mN/m or 60 to 65 mN/m. The surface tension was adjusted if needed by changing the amount of the mentioned above compounds.

A Coronavirus (COVID-19) IgM/IgG Rapid Test Kit (dual cassette) and components, Positive control IgG and IgM solutions and Commercial Diluent, were obtained from RayBiotech (Peachtree, GA). This Rapid Test Kit is suitable for qualitative detection of SARS-CoV-2 (COVID-19) N-Protein IgM and IgG antibodies in human serum, whole blood, or finger prick samples, according to the information from RayBiotech (Peachtree, GA) website. As a Diluent for all series of dilutions the Commercial Diluent from the same Rapid Test Kit was used. The positive COVID-19 standards obtained from RayBiotech (Peachtree, GA) were: 1) CoV-PosG-S-500 COVID-19 IgG positive serum sample; 2) CoV-PosM-S-500 COVID-19 IgM positive serum sample; 3) CoV-PosG-P-500 COVID-19 IgG positive plasma sample; 4) CoV-PosM-P-500 COVID-19 IgM positive plasma sample.

A serial dilution was performed with CoV-PosG-S-500 COVID-19 IgG positive serum sample and CoV-PosM-S-500 COVID-19 IgM positive serum sample. The dilutions cover the range from 1:10 to 1:333, namely: 1:10, 1:25, 1:50, 1:100, or 3:1000.

The specific amount of one of the positive serum samples (i.e., CoV-PosG-S-500 COVID-19 IgG positive serum sample and CoV-PosM-S-500 COVID-19 IgM positive serum sample) was 20 μl for 1:10 dilution; 8 μl for 1:25 dilution; 4 μl for 1:50 dilution; 2.2 μl for 1:100 dilution. For 1:333 dilution, 6 μl of 1:100) were mixed with a specific amount of commercially available Sample Diluent, namely, 180 μl for 1:10 dilution; 192 μl for 1:25 dilution; 196 μl for 1:50 dilution; 217.8 μl for 1:100 dilution; 194 μl for 1:333 dilution.

The test strips were removed from the commercial housing and were combined into 13 sets by 5 test strips (IgG and IgM) in each set. The rest of the test strips were placed into set #14.

A multichannel pipettor was used for simultaneous deposition of about 50 μl of a relevant sample dilution on a sample pad of each of 5 test strips. After that, the 5 test stripes were placed on a holder inside of the ESEQuant LR3 lateral flow reader (DIALUNOX GmbH, Stockach, Germany) for reading the test results by scanning the test strips.

FIGS. 6A-B show the photos of the LFA test strips at about 10 minutes of analysis time. FIG. 6A shows side-by-side comparison of LFA test strips after injection in each of them 50 microliters of 1:25 dilution of standard COVID 19 IgM positive serum control sample: Control sample-negative serum control sample; Commercial 1:25 dilution samples-1:25 dilution of standard COVID 19 IgM positive serum control sample; Modified 1:25 dilution samples-1:25 dilution of standard COVID 19 IgM positive serum control sample in modified approach according to the present invention. Commercial LFA test strips, the commercially available lateral flow rapid test strips without any modification, were used according to the company manual protocol. Modified LFA test strips were the same model of the commercially available lateral flow rapid test strips additionally modified according to the present invention. Photos of the LFA test strips were taken at about 10 minutes of analysis time.

A serial dilution was performed on CoV-PosM-S-500 COVID-19 IgM positive serum sample (FIG. 5). The dilutions covered a range from 1/10 to 3/1000. Improvement for the equilibrium dissociation constants Kd was determined about 1.7 times for the COVID-19 IgM positive serum control samples (Kd-com=0.01187 in dilution units, i.e., the antibody titer of 1:84 and Kd-mod=0.006976 in dilution units, i.e., the antibody titer of 1:143), additionally to significantly improve the visibility (contrast) of the Test Line in modified LFA (data not shown). For preliminary estimation, the ratio between equilibrium dissociation constants Kd-com (commercial LFA) and Kd-mod (improved/modified LFA) was determined from the binding curve fitting by utilizing the model for specific binding (FIG. 5).

Improvement for visibility (contrast) of the LFA test lines was determined in a range from about 10-times at 1:333 IgM COVID-19 positive sample dilution to about 2-times for all other checked IgM COVID-19 positive sample dilutions, namely: 1:10, 125, 1:50, 1:100, when comparing the commercial test strips and modified according to the present invention test strips (FIG. 6B).

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for detecting an analyte in a liquid test sample, comprising:
(a) mixing the test sample with a liquid enhancement composition to make a mixture, wherein the enhancement composition comprises one or more non-ionic amphiphilic polymeric compounds selected from the group consisting of hydroxyethylcellulose (HEC), hydroxypropylcellulose (HPC), methylcellulose (MC), hydroxyethyl methyl cellulose (HEMC), and hydroxypropylmethylcellulose (HPMC) and a combination thereof, wherein the mixture comprises the one or more non-ionic amphiphilic polymeric compounds at a combined concentration of 10-1000 times of the critical aggregation concentration (CAC) of the one or more non-ionic amphiphilic polymeric compounds, and the mixture has a surface tension σ of 55-67 mN/m; and
(b) contacting the mixture with a binding agent capable of forming a complex with the analyte, wherein the presence of the complex of analyte and the binding agent indicates the presence of the analyte in the test sample.

2. The method of claim 1, wherein the enhancement composition further comprises an agent selected from the group consisting of bovine serum albumin, D-fructose, gelatin, pectin, sodium bicarbonate and a combination thereof.

3. The method of claim 2, wherein the mixture further comprises bovine serum albumin at 0.1-0.4 mg/ml based on the total volume of the mixture.

4. The method of claim 2, wherein the mixture further comprises D-fructose at 0.01-0.7 wt % based on the total weight of the mixture.

5. The method of claim 2, wherein the mixture further comprises gelatin at 0.001-0.05 wt % and/or pectin at 0.1-1 wt %, each wt % based on the total weight of the mixture.

6. The method of claim 2, wherein the mixture further comprises sodium bicarbonate at 0.01-0.2 wt % based on the total weight of the mixture.

7. The method of claim 1, wherein the analyte is selected from the group consisting of peptides, proteins, polysaccharides, polymers, and nucleic acids.

8. The method of claim 1, wherein the analyte is an antibody or an antigen.

9. The method of claim 1, further comprising mixing the test sample and the enhancement composition on a mixing material.

10. The method of claim 9, wherein the mixing material comprises interstices, channels, cavities and/or pores.

11. A mixing device comprising:
(a) a first compartment comprising a liquid test sample;
(b) a second compartment comprising a liquid enhancement composition, wherein the enhancement composition comprises one or more non-ionic amphiphilic polymeric compounds selected from the group consisting of hydroxyethylcellulose (HEC), hydroxypropylcellulose (HPC), methylcellulose (MC), hydroxyethyl methyl cellulose (HEMC), and hydroxypropylmethylcellulose (HPMC) and a combination thereof; and
(c) a third compartment in which the test sample from the first compartment and the enhancement composition from the second compartment are mixed to make a mixture, wherein the mixture comprises the one or more non-ionic amphiphilic polymeric compounds at a combined concentration of 10-1000 times of the critical aggregation concentration (CAC) of the one or more non-ionic amphiphilic polymeric compounds, and the mixture has a surface tension σ of 55-67 mN/m.

11. The mixing device of claim 10, wherein the mixing device is selected from the group consisting of pipette, syringe, transfer plastic pipette, pipette droppers, eye dropper, pasteur pipette, disposable pipettes, capillaries and microfluidics.

12. The mixing device of claim 10, wherein the liquid test sample comprises an analyte selected from the group consisting of peptides, proteins, polysaccharides, polymers, nucleic acids.

13. The mixing device of claim 10, wherein the analyte is an antibody or an antigen.

14. The mixing device of claim 11, wherein the third compartment comprises a mixing material, wherein the test sample and the enhancement composition are mixed on the mixing material.

15. The mixing device of claim 14, wherein the mixing material comprises interstices, channels, cavities and/or pores.

16. The method of claim 3, wherein the mixture further comprises D-fructose at 0.01-0.7 wt % based on the total weight of the mixture.

17. The method of claim 3, wherein the mixture further comprises gelatin at 0.001-0.05 wt % and/or pectin at 0.1-1 wt %, each wt % based on the total weight of the mixture.

18. The method of claim 3, wherein the mixture further comprises sodium bicarbonate at 0.01-0.2 wt % based on the total weight of the mixture.

19. The method of claim 2, wherein the analyte is selected from the group consisting of peptides, proteins, polysaccharides, polymers, and nucleic acids.

20. The method of claim 2, wherein the analyte is an antibody or an antigen.
